(19) United States
(12) Patent Application Publication
(10) Pub. No.: US 2004/0116897 A1
(43) Pub. Date: Jun. 17, 2004

(54) Minimally invasive bypass system and related methods

(76) Inventor: Walid Aboul- Hosn, Fair Oaks, CA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/332,627

(22) PCT Filed: Jul. 11, 2001
(86) PCT No.: PCT/US01/21862

(30) Foreign Application Priority Data
Jul. 12, 2000 (US) 60218001

(51) Int. Cl. A61M 31/00; A61M 25/00; A61M 37/00; A61F 11/00; A61B 1/00; A61M 5/00

(52) U.S. Cl. 604/508; 604/264; 604/95.04; 606/108; 600/149

(57) ABSTRACT

A system and related methods for performing minimally invasive vascular bypass surgery, such as coronary artery bypass surgery. The system and methods involve the feature of providing a stabilizer introducer (10) dimensioned for minimally invasive introduction into the chest cavity of a patient. The stabilizer introducer has a central lumen (22) having an open distal end which, when disposed over a surgical site, may be filled with biocompatible fluid to establish a “fluid filled column” within the stabilizer introducer. An incision is then created through the wall of the diseased blood vessel within the surgical site (from outside or inside the blood vessel), through which a bypass conduit may be passed in order to restore flow.

### BACKGROUND OF THE INVENTION

I. Field of the Invention

The present invention relates generally to the field of minimally invasive surgery. More particularly, the present invention relates to a system and related methods for performing minimally invasive vascular bypass surgery, such as coronary artery bypass surgery.

II. Discussion of the Prior Art

Vascular bypass surgery involves reestablishing the flow of blood past an area of restricted flow within a blood vessel. Such restricted blood flow may result from the accumulation of artherosclerotic plaque on the inner walls of blood vessels which, over time, causes a narrowing or occluding of these vessels. In coronary artery disease, the narrowing or occlusion of the coronary arteries is especially threatening in that it results in insufficient blood flow to the heart tissue. With an increasing number of older and elderly patients, coronary artery disease represents one of the most common life-threatening medical problems.

Treatments for coronary artery disease include drugs, interventional devices, and/or bypass surgery. High doses of thrombolytics (clot-dissolving drugs) are frequently used in an effort to dissolve the blood clots. Even with such aggressive therapy, thrombolytics fail to restore blood flow in the affected vessel in about 30% of patients. In addition, these drugs can also dissolve beneficial clots or injure healthy tissue causing potentially fatal bleeding complications.

While a variety of interventional devices are available, including angioplasty, artherectomy, and laser ablation catheters, the use of such devices to remove obstructing deposits may leave behind a wound that heals by forming a scar. The scar itself may eventually become a serious obstruction in the blood vessel (a process known as restenosis). Also, diseased blood vessels being treated with interventional devices sometimes develop vasoconstriction (elastic recoil), a process by which spasms or abrupt reclosure of the vessel occur, thereby restricting the flow of blood and necessitating further intervention. Approximately 40% of treated patients require additional treatment for restenosis resulting from scar formation occurring over a relatively long period, typically 4 to 12 months, while approximately 1-in-20 patients require treatment for vasoconstriction, which typically occurs from 4 to 72 hours after the initial treatment.

Percutaneous transluminal coronary angioplasty (PTCA), also known as balloon angioplasty, is yet another treatment for coronary vessel stenosis. The increasing popularity of the PTCA procedure is attributable to its relatively minimal invasiveness compared with coronary by-pass surgery. Patients treated by PTCA, however, suffer from a high incidence of restenosis, with about 35% of all patients requiring repeat PTCA procedures or by-pass surgery, with attendant high cost and added patient risk. More recent attempts to prevent restenosis by use of drugs, mechanical devices, and other experimental procedures have had limited success.

Restenosis occurs as a result of injury to the arterial wall during the lumen opening angioplasty procedure. In some patients, the injury initiates a repair response that is characterized by hyperplastic growth of the vascular smooth muscle cells in the region traumatized by the angioplasty. The hyperplasia of smooth muscle cells narrows the lumen that was opened by the angioplasty, thereby necessitating a repeat PTCA or other procedure to alleviate the restenosis.

In typical PTCA procedures, a guiding catheter is percutaneously introduced into the cardiovascular system of a patient and advanced through the aorta until the distal end is in the ostium of the desired coronary artery. Using fluoroscopy, a guide wire is then advanced through the guiding catheter and across the site to be treated in the coronary artery. A balloon catheter is advanced over the guide wire to the treatment site. The balloon is then expanded to reopen the artery.

To help prevent arterial closure, repair dissection, or prevent restenosis, a physician can implant an intravascular prosthesis, or a stent, for maintaining vascular patency inside the artery at the lesion. The stent may either be a self-expanding stent or a balloon expandable stent. For the latter type, the stent is often delivered on a balloon and the balloon is used to expand the stent. The self-expanding stents may be made of shape memory materials such as nitinol or constructed of other metals but of a design which exhibits self expansion characteristics.

In certain known stent delivery catheters, a stent and an optional balloon are positioned at the distal end of the catheter, around a core lumen. The stent and balloon are held down and covered by a sheath or sleeve. When the distal portion is in its desired location of the targeted vessel the sheath or sleeve is retracted to expose the stent. After the sheath is removed, the stent is free to self-expand or be expanded with a balloon.

Coronary artery bypass grafting (CABG) procedures are typically performed by splitting the sternum and pulling open the chest cavity to provide access to the heart. An incision is made in the artery adjacent to the blocked area. The internal mammary artery (IMA) is then severed and attached to the artery at the point of incision. The IMA bypasses the blocked area of the artery to again provide a full flow of blood to the heart. Splitting the sternum and opening the chest cavity (“open chest” surgery) can create a tremendous trauma to the patient. Moreover, the cracked sternum prolongs the recovery period of the patient.

There have been attempts to perform CABG procedures without opening the chest cavity. Minimally invasive procedures are conducted by inserting surgical instruments and an endoscope through a small incision in the chest of the patient. Manipulating such instruments can be awkward, particularly when suturing a graft to a small artery. A high level of dexterity is required to accurately control the instruments, which can be challenging for the surgeon. Robotic instrumentation has been developed for minimally invasive procedures; however, such devices have proven both prohibitively expensive and difficult to control in order to perform surgery in a timely manner.

Although open-chest CABG procedure has become relatively common, the procedure itself is lengthy and traumatic and can damage the heart and cardiovascular system, the central nervous system, and the blood supply. The surgeon must make a long incision down the center of the chest and then cut through the entire length of the sternum. Several other procedures are necessary to attach the patient to a heart-lung bypass machine, stop the blood flow to the heart, and then stop the heart from beating in order to install the graft. The lengthy surgical procedures are necessary, in part, to connect the patient to a cardiopulmonary bypass machine to continue the circulation of oxygenated blood to the rest of the body while the bypass graft is sewn into place.

Although several efforts have been made to make the CABG procedure less invasive and less traumatic, most techniques still require cardiac bypass and cardioplegia (stoppage of the heart). The safety and efficacy of CABG surgery could be improved if the heart could remain beating during the procedure, thereby eliminating cardiopulmonary bypass and the lengthy and traumatic surgical procedures necessary to connect the patient to a cardiopulmonary bypass machine to sustain the patient's life during the procedure. In recent years, a small number of surgeons have begun performing so called “beating heart” CABG procedures using surgical techniques especially developed so that the CABG procedure could be performed while the heart is still beating. In such procedures, there is no need for any form of cardiopulmonary bypass, no need to perform the extensive surgical procedures necessary to connect the patient to a cardiopulmonary bypass machine, and no need to stop the heart.

Despite the advantages, beating-heart CABG surgery is not widely practiced, in part, because of the difficulty in performing the necessary surgical procedures on posterior heart vessels which require manipulating the heart for exposure. In addition, surgeons typically perform such beating-heart procedures through an open chest, which is a major source for patient morbidity.

As noted above, CABG surgery requires that a fresh source of blood be routed past the area of narrowing or occlusion in a coronary artery to thereby restore blood flow to the heart. The connection between the bypass graft and the artery is known as an “anastomosis.” Typically, one end of the bypass graft is sewn to a source artery with an unobstructed blood flow, such as the left internal mammary artery (LIMA). The other end of the bypass graft is sewn to a target coronary artery downstream from the occlusion, such as the left anterior descending artery (LAD). In this fashion, blood flow is restored to the main muscles of the heart. Because the beating-heart CABG procedure is performed while the heart muscle is continuing to contract and pump blood, performing the aforementioned anastomosis procedure is difficult to perform because the heart continues to move and to attempt to pump blood while the surgeon is sewing the graft in place. The surgical procedure necessary to install the graft in the beating-heart CABG procedure requires placing a series of sutures through several extremely small vessels that continue to move during the procedure. Moreover, the sutures must be carefully placed so that the graft is firmly attached and does not leak when blood flow through the graft is established.

Another drawback of traditional bypass grafting is that the procedure must be performed rapidly because the blood flow through the target coronary artery is interrupted or reduced during the procedure to allow the graft to be installed without excessive blood loss. Furthermore, the working space and visual access are limited because the surgeon may be working through a small incision in the chest or may be viewing the procedure on a video monitor if the site of the surgery is viewed via a surgical scope.

### SUMMARY OF THE INVENTION

The present invention overcomes the above-identified drawbacks of the prior art techniques for treating vascular restrictions due to blood vessel disease. The present invention accomplishes this by providing a system and related methods for minimally invasive vascular bypass procedures by means of a simplified procedure for establishing the necessary bypass. The present invention is superior to present methods because: (1) it does not require an open the chest; (2) the elapsed time needed to complete the procedure is reduced, thereby reducing the recovery time; and (3) it eliminates the need for invasive medical procedures, thereby reducing trauma to the patient.

The present invention provides an introducer that may be inserted in the patient's chest to gain access to the heart or other organs. The introducer has a flexible elongated hollow body having a generally cylindrical shape. The introducer provides hemostasis control of the surgical site, and alternatively may be utilized to stabilize the surgical site. The stabilizer introducer may be advanced to the pre-selected surgical site and fixedly attached to the surface of the heart, thereby isolating and stabilizing the site. The stabilizer introducer is designed to provide hemostasis control of the surgical site, as well as to introduce surgical instruments and/or devices (such as stents) to the site. The stabilizer introducer may have a main lumen, which can be utilized to enhance visibility of the surgical site. The stabilizer introducer is designed so that, in use, the stabilization force applied to the tissue is directly applied to the surgical site. The stabilizer introducer of the present invention may be constructed of multiple pieces which when combined create a hollow elongated tube having selectable rigidity.

### BRIEF DESCRIPTION OF THE DRAWINGS

The following description of the preferred embodiments of the present invention will be better understood in conjunction with the appended drawings, in which:

FIG. 1 is a side view (partially in section) illustrating a stabilizer introducer for minimally invasive vascular bypass surgery according to one embodiment of the present invention disposed over a diseased coronary artery on the anterior of the heart;

FIG. 2 is a side view of the stabilizer introducer as shown in FIG. 1;

FIG. 3 is a cross-sectional view of the stabilizer introducer shown in FIG. 2 taken along a longitudinal plane;

FIG. 4 is a cross-sectional view of the stabilizer introducer taken along lines 4-4 in FIG. 2;

FIG. 5 is an end view of the stabilizer introducer as viewed from lines 5-5 in FIG. 2;

FIG. 6 is a partial sectional view illustrating steps in performing minimally invasive cardiac surgery using the stabilizer introducer of the present invention, namely positioning the stabilizer introducer over a diseased area of a blood vessel, and disposing a catheter within the diseased area;

FIG. 7 is a partial sectional view illustrating a subsequent step in performing minimally invasive cardiac surgery using the stabilizer introducer of the present invention, namely filling the central lumen of the stabilizer introducer with fluid to establish a “fluid filled column”;

FIG. 8 is a partial sectional view illustrating a still further step in performing minimally invasive cardiac surgery using the stabilizer introducer of the present invention, namely creating an incision through the wall of the blood vessel (from the outside) over at least part of the diseased area;

FIG. 9 is a partial sectional view illustrating a an alternate technique for creating an incision through the wall of the blood vessel, namely from the interior of the blood vessel through the use of a catheter having at least one cutting element;

FIG. 10 is a partial sectional view illustrating a final step in performing minimally invasive cardiac surgery using the stabilizer introducer of the present invention, namely deploying a bypass conduit (such as a stent) within the diseased region;

FIG. 11 is a side view (partially in section) illustrating a stabilizer introducer for minimally invasive vascular bypass surgery according to another embodiment of the present invention disposed over a diseased coronary artery on the anterior of the heart;

FIG. 12 is a side view (partially in section) illustrating a stabilizer introducer of the type shown in FIG. 11 disposed over a diseased coronary artery on the posterior of the heart;

FIGS. 13 and 14 are side and perspective views, respectively, of the stabilizer introducer shown in FIGS. 11 and 12;

FIG. 15 is a cross-sectional view of the stabilizer introducer shown in FIG. 13 taken along a longitudinal plane;

FIGS. 16 and 17 show the details of segments that form the elongated body of the stabilizer introducer shown in FIGS. 11-15.

FIGS. 18 and 19 are side and perspective views, respectively, of a stabilizer introducer of the type shown in FIGS. 11-15 having a lateral distal opening;

FIG. 20 is a side view (partially in section) illustrating a stabilizer introducer of the type shown in FIGS. 11-15 having supplemental stabilizing members extending laterally from the distal opening;

FIG. 21 is a side view of the stabilizer introducer shown in FIG. 20;

FIG. 22 is a perspective view of a stabilizer introducer of the type shown in FIGS. 18 and 19 having supplemental stabilizing members;

FIGS. 23 and 24 are side and cross-sectional views, respectively, of a stabilizer introducer according to a further embodiment of the present invention having a control feature for controlling the orientation of the distal opening;

FIGS. 25 and 26 are perspective and side views, respectively, of a stabilizer introducer for minimally invasive vascular bypass surgery according to a still further embodiment of the present invention having an introducer extending longitudinally through a stabilizer; and

FIG. 27 is a cross-sectional view of the stabilizer introducer shown in FIGS. 25 and 26 taken along a longitudinal plane.

### DESCRIPTION OF THE PREFERRED EMBODIMENTS

Illustrative embodiments of the present invention are described below. In the interest of clarity, all features of an actual implementation may not be described in this specification. It will of course be appreciated that in the development of any such actual embodiment, numerous implementation-specific decisions must be made to achieve the developers' specific goals, such as compliance with business-related constraints, which may vary from one implementation to another. Moreover, it will be appreciated that such a development effort might be complex and time-consuming, but would nevertheless be a routine undertaking for those of ordinary skill in the art having the benefit of this disclosure.

The present invention provides a system and method for performing minimally invasive beating heart surgery, which involves deploying a bypass conduit (such as a stent) through an incision in the wall a diseased blood vessel. A device having the combined features of a stabilizer and introducer is employed to stabilize the diseased region of the blood vessel and establish a fluid-filled lumen through which to introduce surgical instruments and devices to the surgical site. This system and method is advantageous over the prior art in that it reduces trauma on the patient, reduces the time required to perform the procedure, and provides the surgeon with improved visual access to the surgical site.

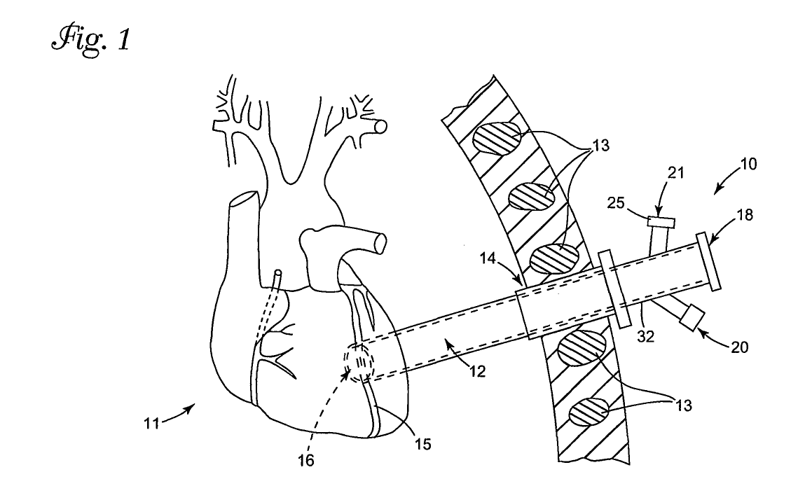

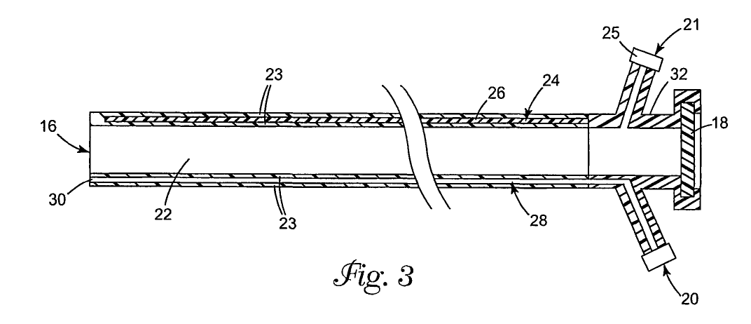

FIG. 1 illustrates a stabilizer introducer 10 according to one embodiment of the present invention. The stabilizer introducer 10 includes an elongated body 12 extending into the chest of the patient through a chest access port 14. The chest access port 14 is shown in FIG. 1 extending through thoracic ribs 13. The elongated body 12 has a distal opening 16 disposed, by way of example, over a diseased region of a coronary artery 15 on the anterior of the heart 11. The proximal end of the elongated body 12 is equipped with a housing member 32 having hemostasis valve assembly 1 8, a suction port assembly 20, and a fluid port assembly 21. As best shown in FIGS. 3-5, a central lumen 22 within the elongated body 12 provides a workspace for the purpose of introducing surgical instruments and devices to the surgical site. Such instruments and devices may be advanced to the surgical site by first passing them through the hemostasis valve 18 and then onward down the length of the lumen 22 to the distal opening 16. The hemostasis valve assembly 18 may be a silicon diaphragm with one or more openings that form a seal around instruments and devices entered through one of the openings in the valve assembly 18 and into the central lumen 22. The central lumen 22 is preferably capable of receiving fluid (such as saline or CO₂) therein so as to establish a “fluid-filled column” within the stabilizer introducer 10. For purposes of this patent, the term “fluid” will be understood to include gasses such as CO₂. The fluid may be received through the fluid port assembly 21 using a syringe (not shown) to inject the fluid into the fluid port assembly 21 and thus the central lumen 22. The fluid port assembly 21 may have a twist valve 25. The twist valve 25 is twisted open to allow fluid to be injected into the central lumen 22, and then twisted closed. Such a “fluid-filled column” is advantageous in that it provides the surgeon with a clear visual access to the surgical site, as well as prevents embolisms from being introduced into the incision created in the blood vessel.

Stabilizer introducer 10 may be constructed of any biocompatible materials such as metal or plastic. In a preferred embodiment, the stabilizer introducer 10 is constructed using a silicone-based plastic. Elongated body 12 may be formed as a unitary body (as shown in FIGS. 1-5) or, alternatively, may be formed of multiple pieces joined together with a biocompatible adhesive or similar joining methods. Hemostasis valve 18 may comprise any number of devices (including those commercially available) for maintaining hemostasis during the introduction and removal of instruments into and from the fluid-filled lumen 22. In similar fashion, chest access port 14 may comprise any number of devices (including those commercially available) for establishing port access into the chest of a patient.

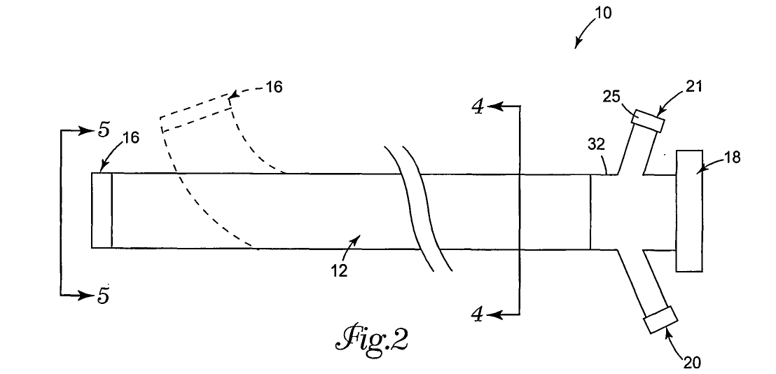

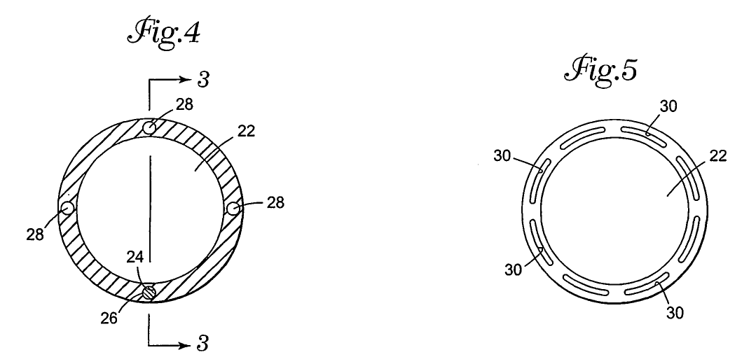

A plurality of additional lumens may be provided within the wall 23 of the elongated body 12. For example, as shown in FIGS. 3 and 4, a lumen 24 may be provided to receive a stiffening member 26. The stiffening member 26 is preferably constructed of a biocompatible material having sufficient pliability and rigidity such that a surgeon may manually alter the shape of the elongated body 12 for the purpose of properly orienting the distal opening 16 against the heart (see FIG. 2). One or more lumen 28 may be provided within the wall of the elongated body 12 for the purpose of establishing fluid communication between the suction port 20 and a plurality of suction ports 30 disposed radially outward from the distal opening 16. The suction port 20 is preferably coupled to a vacuum source (not shown) for the purpose of creating a suction force to causes the suction ports 30 to be drawn against the heart tissue surrounding the surgical site. This advantageously aids in stabilizing the heart tissue local to the surgical site, as well as in maintaining a seal between the distal end of the elongated body 12 and the heart tissue to prevent the ingress or egress of fluid into or out of the central lumen 22.

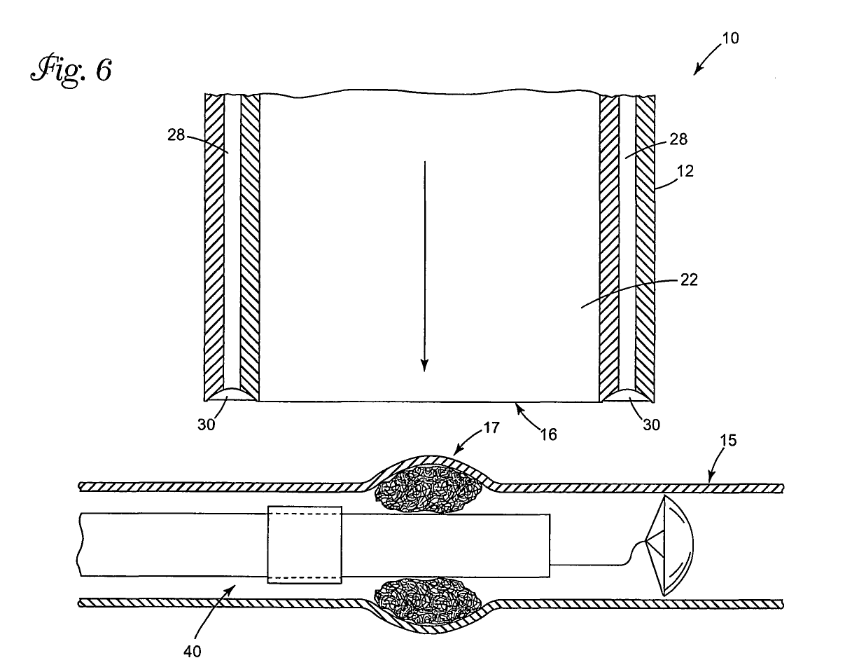

The method of performing minimally invasive beating heart surgery according to one embodiment of the present invention will now be described in detail with reference to FIGS. 6-10. With reference to FIG. 6, the stabilizer introducer 10 is advanced into the chest of the patient such that the distal opening 16 is disposed over the diseased region 17 of the subject vessel 15. This may be facilitated through the use of the stiffening rod 26 discussed above. That is, a surgeon may easily bend the elongated body 12 (as shown in FIG. 2) as necessary to ensure the distal opening 16 is properly oriented over the diseased region 17 of the blood vessel 15. Positioning the distal opening 16 may be aided by using an endoscope (not shown) that is placed in the central lumen through a hole in the hemostasis valve assembly 18. As will be discussed in greater detail below, a catheter 40 may also be advanced into the diseased region 17 to aid in creating an incision in the blood vessel 15 over at least part of the diseased region 17. The catheter 40 is advanced into this position by first introducing it into the vasculature at a remote location, such as the femoral artery or similar artery that is in fluid communication with diseased region 17. Fluoroscopy or similar techniques may be employed to facilitate placement of the catheter 40.

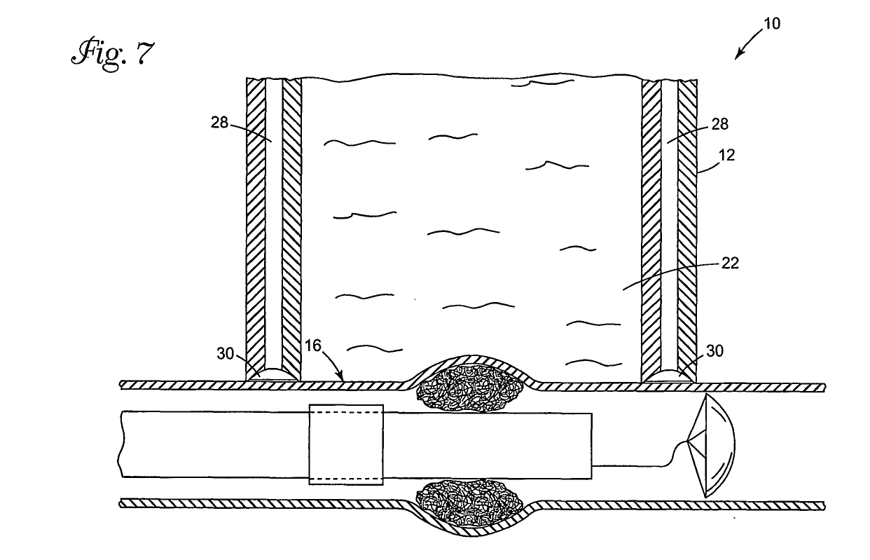

As shown in FIG. 7, the next step involves securing the distal opening 16 of the stabilizer introducer 10 in position about the surgical site. This is preferably accomplished by positioning the distal opening 16 against the heart tissue and activating the vacuum source (not shown, but attached to suction port assembly 20 shown in FIGS. 1-3) to create suction at the suction ports 30. With the distal opening 16 in position, the lumen 22 may thereafter be filled with any of a variety of fluids, including but not limited to saline or carbon dioxide. The fluid may be introduced at a pressure greater than the vasculature. The pressure level may be adjusted, using fluid port assembly 21, to ensure that, after an incision of the vessel 15 (which will be described later) the fluid in lumen 22 does not enter the vessel 15 and blood from the vessel 15 does not exit into lumen 22. Saline, for example, has little or no oxygen content, and so it may be preferable to avoid the entry of saline into the vessel 15. In addition, blood exiting the vessel 15 into the lumen 22 may obscure the surgeon's view of the surgical site. By filling and pressurizing lumen 22 with fluid in this fashion, a clear view of the surgical site is created, thereby allowing the surgeon to perform the procedure under more favorable conditions. This also reduces, if not eliminates, the chance of an embolism being introduced into the patient's vasculature during the procedure.

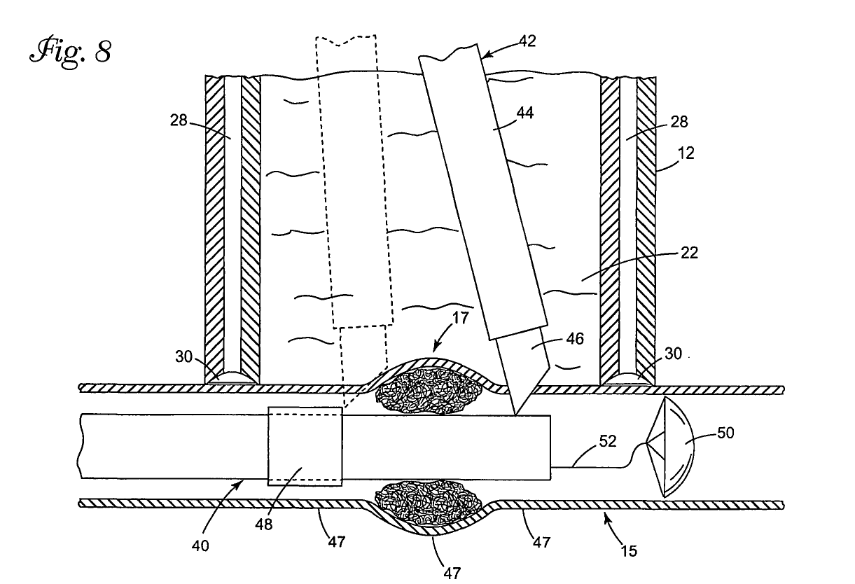

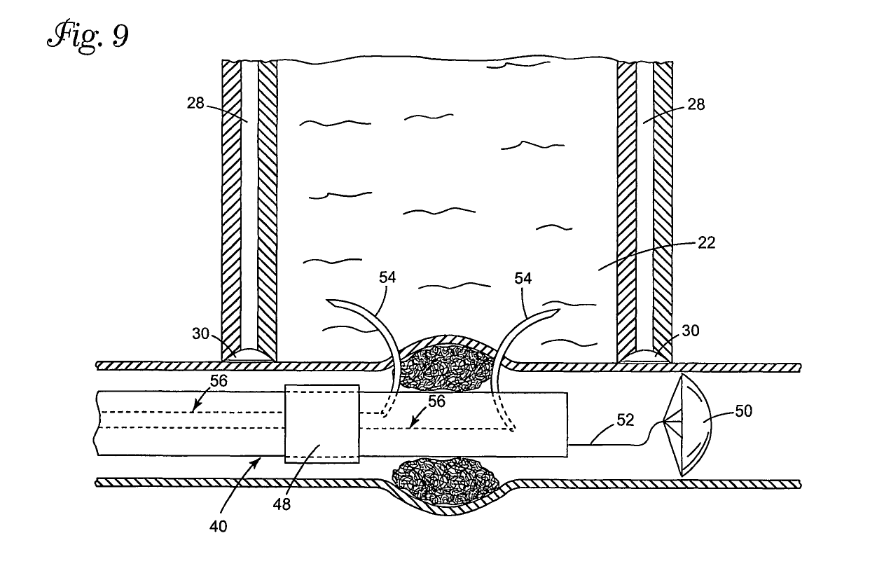

The next step in the method of the present invention involves creating an incision through the wall of the blood vessel 15 over at least part of the diseased region 17. This may be accomplished from outside the blood vessel 15 (i.e. FIG. 8) or inside the blood vessel 15 (i.e. FIG. 9). With reference to FIG. 8, an incision may be created from the exterior of the blood vessel by introducing a cutting device 42 through the central lumen 22. The cutting device 42, by way of example only, may include a handle 44 having a blade 46 extending therefrom. The handle 44 may be manipulated by the surgeon such that the blade 46 is caused to pierce the wall of the blood vessel (as shown in phantom) and progressed longitudinally (left to right in FIG. 8) to create an incision at least partially over the diseased region. The catheter 40 provides a protective barrier to ensure that the blade 46 will not pierce the opposite wall 17 of the blood vessel 15.

The catheter 40 may preferably be equipped with a balloon 48 and a debris catcher 50. The balloon 48 may assist in placing the catheter 40 in position, such as by using it as a “sail” within the blood stream to advance it to the surgical site. The balloon 48 may also aid in maintaining the catheter 40 in position during the procedure, such as by inflating the balloon 48 into abutment with the vessel 15 wall after the catheter 40 has been properly positioned through the diseased region 17. The balloon 48 may also be employed to deploy various bypass conduits after they have been introduced into the diseased region 17 through the incision. The debris catcher 50 may comprise any number of materials or structures capable of receiving debris dislodged during the cutting procedure and preventing such debris from continuing downstream through the blood vessel 15. In one embodiment, the debris catcher 50 may be deployed from a lumen (not shown) formed within the wall of the catheter 40 and retracted following use by selectively advancing and withdrawing a wire or line 52 coupled to the debris catcher 50.

With reference to FIG. 9, an incision may alternatively be created from the interior of the blood vessel 15 by equipping the catheter 40 with one or more cutting elements 54. In one embodiment, the cutting elements 54 may be selectively manipulated from a retracted state (not shown) within the catheter 40 to the deployed state shown by pulling internally disposed wires or rods 56. In this fashion, the surgeon may easily create the incision by simply pulling the wires 56 and then retract the cutting elements 54 prior to withdrawing the catheter 40 by advancing the wires 56 to return the cutting elements 54 to their retracted position. The cutting elements 54 may be provided in any number of different configurations, including but not limited to the sickle or curved shape shown.

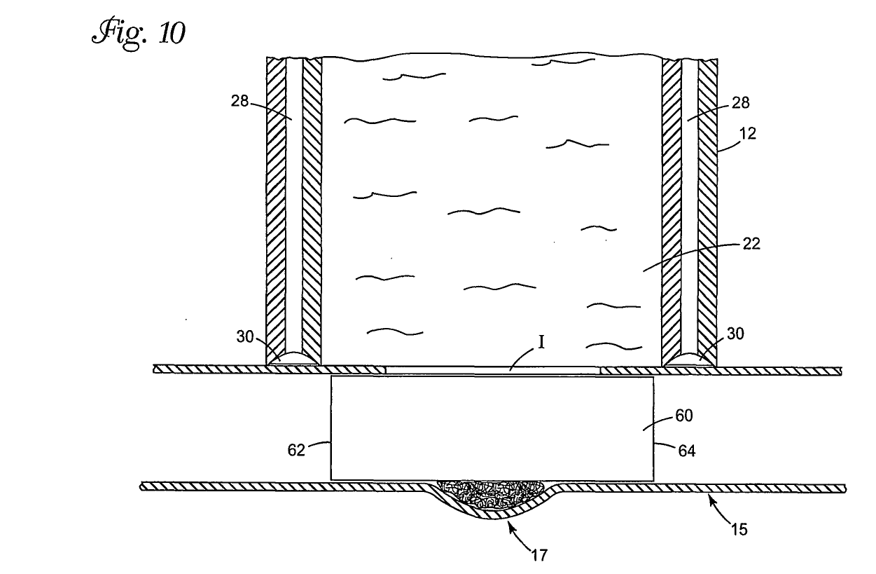

FIG. 10 illustrates the next step in the method of performing minimally invasive cardiac surgery of the present invention, namely positioning a bypass conduit 60 through the diseased region 17 of the blood vessel 15. This step is accomplished by introducing the bypass conduit 60 through the incision (I) formed in the wall of the blood vessel and manipulating the bypass conduit 60 such that its first end 62 is disposed upstream from diseased region 17 and its second end 64 is disposed downstream from the diseased region 17. The bypass conduit 60 may comprise any number of suitable conduits for reestablishing adequate blood flow past the diseased region, including but not limited to stents, synthetic grafts, autologous grafts harvested from the patient's own vasculature, and grafts tissue engineered from the patient's own DNA. In one embodiment, the bypass conduit 60 may comprise a graft arrangement of the type shown and described in commonly-owned U.S. Provisional Patent Application Ser. No. 60/262,742, entitled “Apparatus for Maintaining Flow Through A Vessel or Duct”, filed Jan. 19, 2001 under Express Mail Label No. EF089158435US, the contents of which are hereby expressly incorporated by reference into this disclosure as if fully set forth herein. Following the introduction of the bypass conduit 60, the incision (I) may be sewn shut by the surgeon (particularly if the bypass conduit 60 is porous, such as a standard stent) or may be left open (particularly if the bypass conduit 60 is non-porous, such as the lined stent systems described in U.S. Provisional Patent Application No. 60/262,742 mentioned above). In either event, the stabilizer introducer 10 may then be withdrawn. The bypass conduit 60 will thus provide a restored blood flow past the diseased region 17.

Removal of the fluid from the central lumen 22 may be done either before or after the stabilizer introducer 10 is withdrawn. To remove the fluid before withdrawal of the stabilizer introducer 10, the fluid may be suctioned out of the central lumen 22 using fluid port assembly 21. While the fluid is being suctioned out of the central lumen 22, ambient air may at the same time be entering the central lumen 22 through, for example, the hemostasis valve assembly 18.

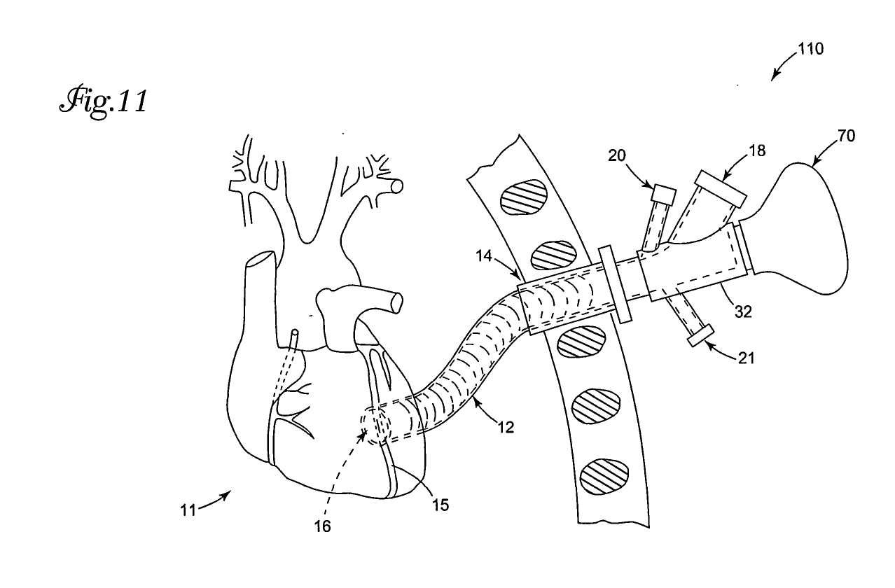

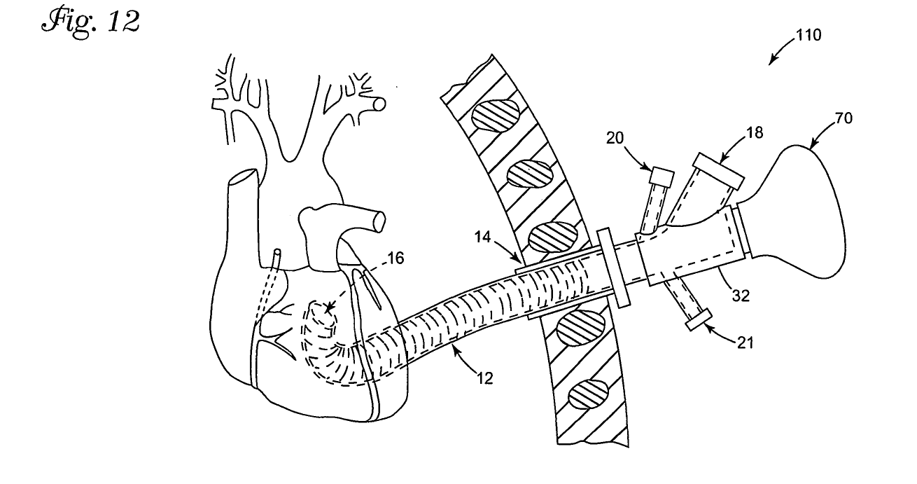

FIGS. 11 and 12 illustrate a stabilizer introducer 110 according to another embodiment of the present invention. The stabilizer introducer 110 differs only slightly from the stabilizer introducer 10 described above such that, in the interest of clarity, like elements will be denoted with like reference numerals. In this embodiment, the elongated body 12 is constructed from a plurality of individual segments forming a jointed, articulated tubular assembly. In use, the tubular body 12 extends into the patient's chest by passing through the chest access port 14. As will be explained in greater detail below, the shape of the tubular body 12 may be selectively adjusted (using a control mechanism 70 extending from the hemostasis valve 18) such that the distal opening 16 may be disposed, by way of example, over a diseased region of a coronary artery 15 on the anterior of the heart 11 (FIG. 11) or on the posterior of the heart 11 (FIG. 12).

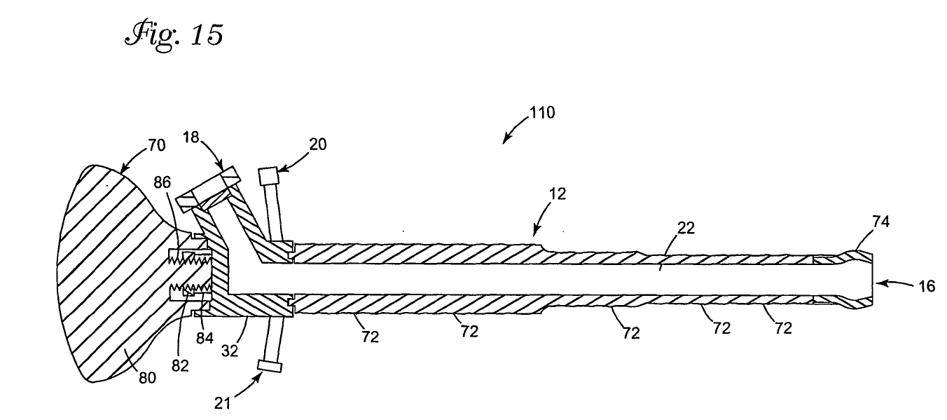

With the exception of the elongated body 12 and the control mechanism 70, the embodiment shown in FIGS. 11-15 operates in entirely the same fashion as the embodiment described above with reference to FIGS. 1-10. That is, with reference to FIG. 15, the central lumen 22 within the elongated body 12 provides a workspace for the purpose of introducing surgical instruments and devices to the surgical site. Once again, this may be accomplished by passing such instruments and devices through the hemostasis valve 18 and then onward down the length of the lumen 22 to the distal opening 16. The central lumen 22 is capable of receiving fluid (such as saline) so as to establish a “fluid-filled column” within the stabilizer introducer 10. As noted above, this feature is advantageous in that it provides the surgeon with a clear visual access to the surgical site, as well as prevents embolisms from being introduced into the incision created in the blood vessel. Stabilizer introducer 110 may be constructed of any biocompatible materials such as metal or plastic. In a preferred embodiment, the stabilizer introducer 110 is constructed using medical grade stainless steel.

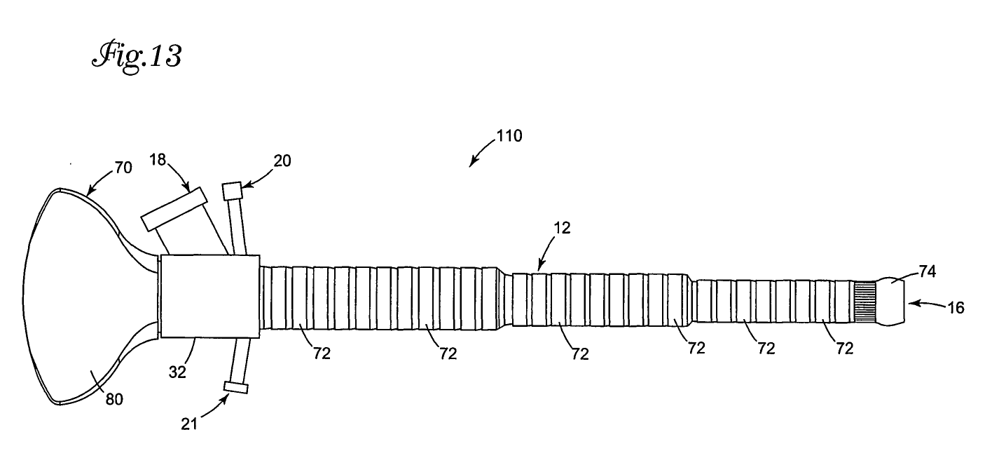

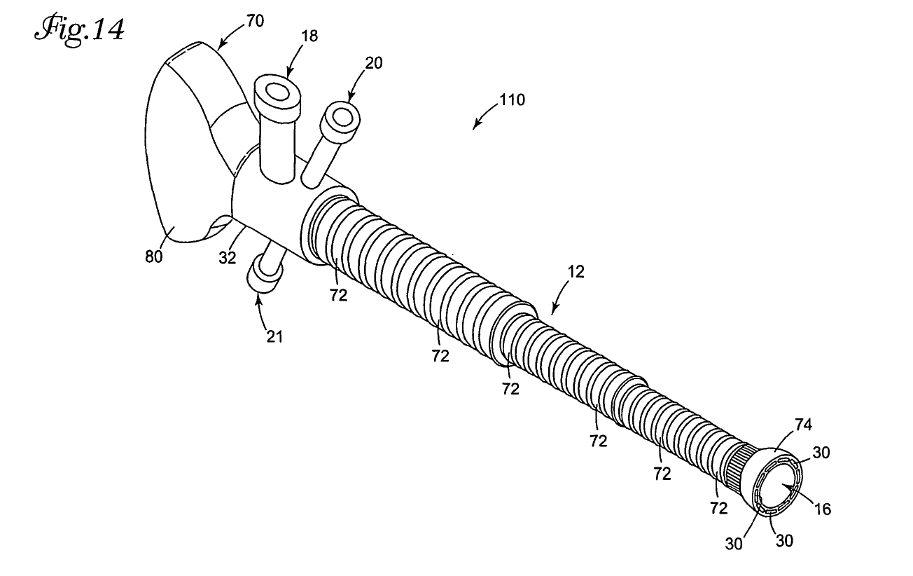

The construction of the elongated body 12 and control mechanism 70 will now be described in detail. As shown in FIGS. 13-15, the elongated body 12 of the stabilizer introducer 110 comprises a plurality of individual segments 72 (not all segments 72 being labeled in FIGS. 13-15) coupled together in a jointed fashion and terminating with a distal member 74. The distal member 74 is configured, by way of example only, in a flared fashion such that the distal opening 16 is slightly larger than the diameter of the central lumen 22. By providing the distal member 74 having this flared configuration, access to the surgical site is maximized, thereby facilitating the surgeon's ability to conduct the surgical procedure of the present invention. The segments 72 forming the elongated body 12 are configured, by way of example, having outer diameters which decrease in a tiered fashion as they progress distally. This tiered construction is advantageous in that it provides bolstered structural integrity of the elongated body 12 when the control mechanism 70 is tightened. The inner diameters of the segments 72 may be generally uniform as shown in FIG. 15, or may be varied along the length of the elongated body 12 depending upon the application. In either case, the segments 72 are provided (either by precise machining or via an internal lining) such that the central lumen 22 is capable of maintaining a “fluid filled column” according to the present invention in a substantially leak-free fashion. A plurality of lumens (not shown) may be provided within the walls of the segments 72 and the distal member 74 to house wire members (forming part of the control mechanism 70 described below) as well one or more fluid conduit(s) extending between the suction port 18 and the suction ports 30 (FIG. 14) on the distal member 74.

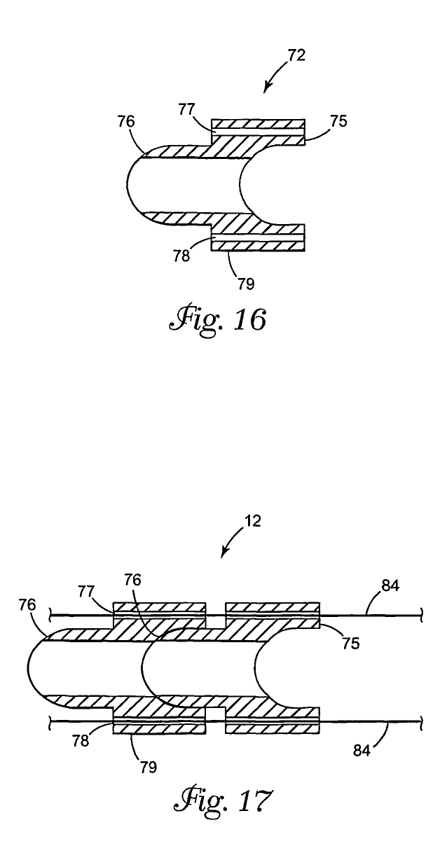

As illustrated in FIG. 16, a segment 72 has a first end 75 and a second end 76. The first end 75 is adapted to slideably couple with second end 76. Segment 72 contains lumens 77 and 78 disposed within wall 79 of segment 72. As shown in FIG. 17, first end 75 is adapted to receive second end 76. Also, by passing cables 84 through lumens 77 and 78, multiple segments may be combined to form elongated body 12. Similar to lumens 77 and 78, but not shown in FIGS. 16-17, walls 79 of segments 72 may have additional lumens corresponding to lumens 28 in FIGS. 3-4 to provide suction at suction ports 30 (FIG. 14) to seal the distal member 75 to the surgical site.

The control mechanism 70 includes a handle member 80, a slide member 82, and one or more cables or wires 84 which extend through lumens (not shown) formed within the walls of the segments 72 and terminate at or near the distal member 74. The handle member 80 includes a centrally disposed threaded member 86 upon which the slide member 82 is threadably engaged. The proximal ends of the cables 84 are fixedly coupled to the slide member 82 such that, by selectively twisting the handle member 80, the slide member 82 may be caused to travel proximally or distally along the threaded member 86. This action of the slide member 82 will, in turn, cause the cables 84 to go into a relaxed or tightened state. In the relaxed state, the joints between the individual segments 72 will become loose and thus allow the elongated body 12 to be adjusted in shape. In the tightened state, the joints between the segments 72 will become fixed and thus allow the elongated body 12 to maintain a rigid state. This rigidity is particularly important in stabilizing the surgical site during beating heart surgery.

The method of performing minimally invasive beating heart surgery using the stabilizer introducer 110 is essentially the same as described above with regard to the stabilizer introducer 10. The stabilizer introducer 110 is advanced into the chest of the patient such that the distal opening 16 is disposed over the diseased region 17 of the subject vessel 15. This may be facilitated by selectively shaping the elongated body 12 while in the relaxed state, fixing the shape thereafter via the control mechanism 70, and then passing the elongated body 12 through the chest access port 14 until the distal member 74 comes to rest over the diseased region. It is also possible to introduce the elongated body 12 into the patient's chest while in a relaxed state, guiding the distal member 74 to a point at or near the target surgical site, and subsequently activating the control mechanism 70 to rigidly maintain the elongated body 12 in that shape. As discussed above, a catheter (such as catheter 40 of FIGS. 6-9) may be advanced into the diseased region to aid in creating an incision in the blood vessel over at least part of the diseased region 17.

With the distal member 74 in position over the target surgical site, the distal opening 16 may then be secured about the surgical site, such as by activating the vacuum source (not shown, but attached to suction port assembly 20 shown in FIGS. 12-15) to create suction at the suction ports 30. The lumen 22 may thereafter be filled with any of a variety of fluids to create a “fluid filled column” therein. As noted above, this advantageously provides a clear view of the surgical site and reduces, if not eliminates, the chance of an embolism being introduced into the patient's vasculature during the procedure. After establishing the “fluid filled column” within the central lumen 22, an incision may be created through the wall of the blood vessel over at least part of the diseased region. This may be accomplished from outside the blood vessel (such as shown in FIG. 8) or inside the blood vessel (such as shown in FIG. 9).

With the incision created through the vessel wall, a bypass conduit (such as the bypass conduit 60 shown in FIG. 10) may then be introduced therethrough and manipulated such that its ends are disposed upstream and downstream from diseased region. Following the introduction of the bypass conduit, the incision may be sewn shut by the surgeon or may be left open depending upon whether the bypass conduit is porous or non-porous. The stabilizer introducer 110 may then be withdrawn, leaving the bypass conduit to provide a restored blood flow past the diseased region.

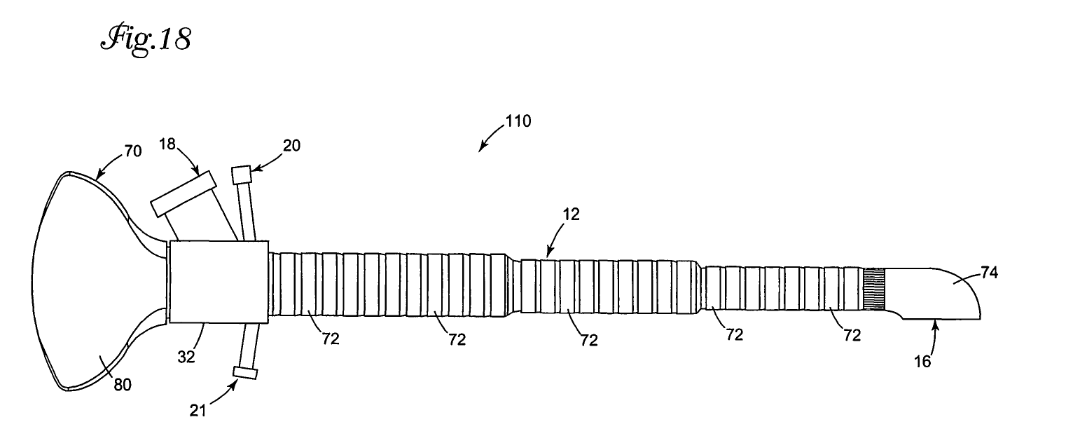

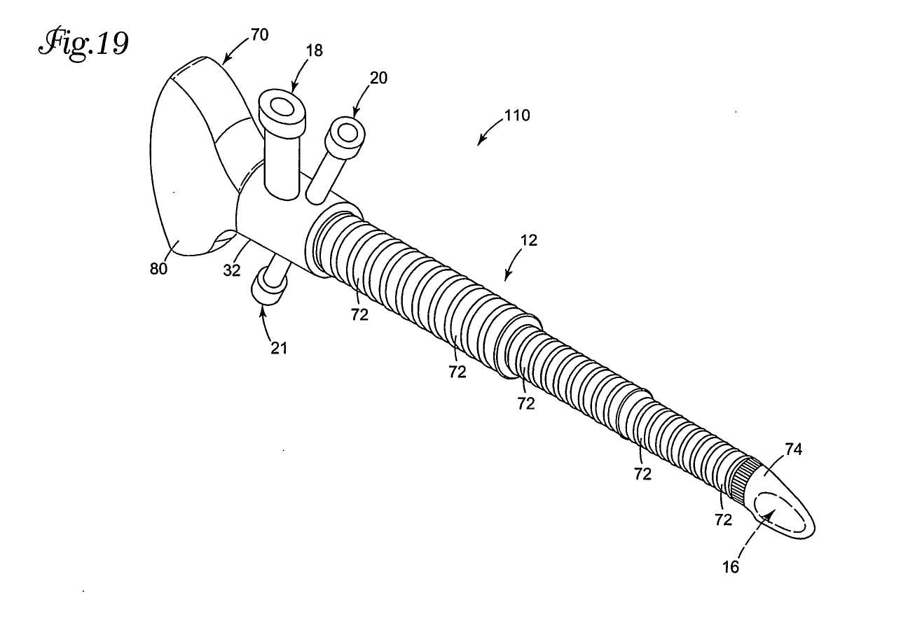

The stabilizer introducer 110 may be equipped with a plurality of differing features without departing from the scope of the present invention. For example, with reference to FIGS. 18 and 19, the distal member 74 of the stabilizer introducer 110 may be provided such that the plane of the distal opening 16 is disposed in a generally perpendicular orientation relative to the longitudinal axis of the central lumen 22. This may be particularly advantageous in establishing surgical access at a point along the lateral or posterior regions of the heart. This may be facilitated by introducing the elongated body 12 through an access port 14 placed in the xyphoid region of the chest. An advantage of utilizing such an access position is that the surgeon can more easily access the apex and/or the anterior or posterior surface of the heart without having to rotate the heart within the patient's chest cavity.

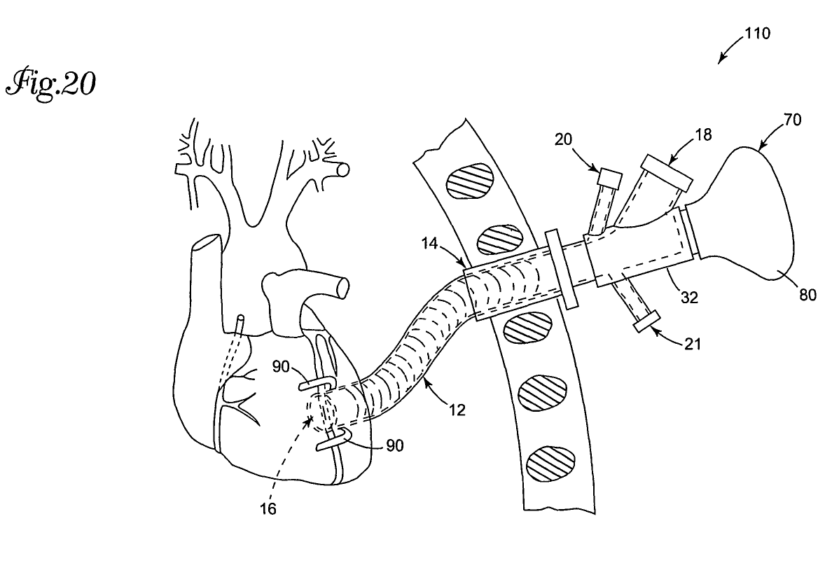

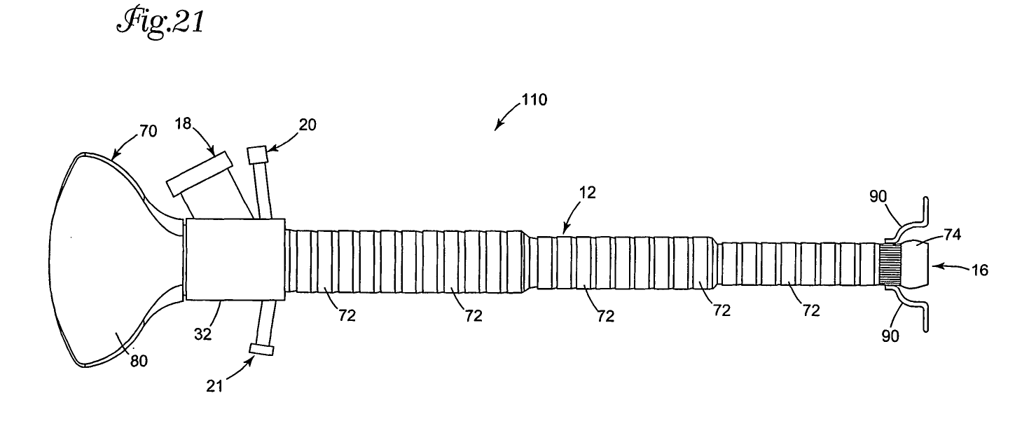

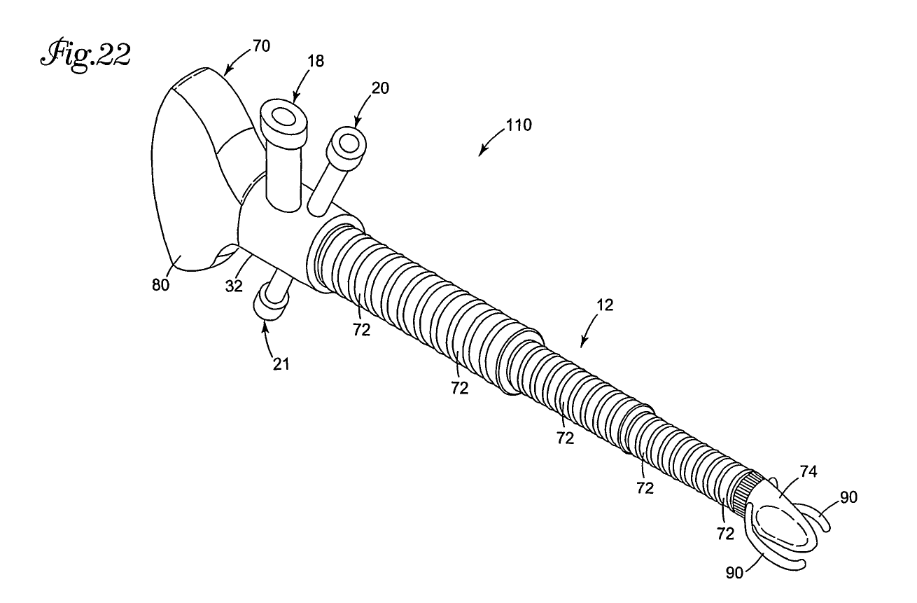

The stabilizer introducer 110 may also be equipped with any of a variety of additional stabilizing features. For example, with reference to FIGS. 20-22, supplemental stabilizing members 90 may be provided extending generally laterally from the distal member 74 for the purpose of providing additional stability when disposed against the tissue of the heart. This stabilizing feature may be augmented by providing additional suction ports (not shown) along the contact surfaces of the stabilizing members 90. Lumens (not shown) extending through stabilizing members 90 may be coupled to a lumen (also not shown in FIGS. 20-22, but similar to lumens 28 shown in FIGS. 3-4) extending in the wall of the elongated member 12 to suction port assembly 20. By coupling these additional suction ports (not shown) to the suction port 20, an additional vacuum force may be exerted against the heart tissue so as to assist in maintaining the distal member 74 firmly in position over the surgical site. As best shown in FIG. 20, the stabilizing members 90 may also aid in cutting off the supply of blood into and out of the blood vessel so as to create a bloodless surgical site.

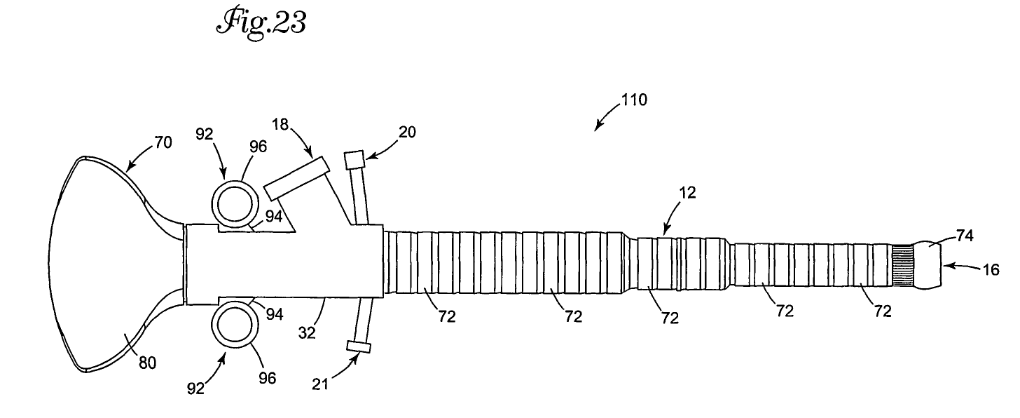

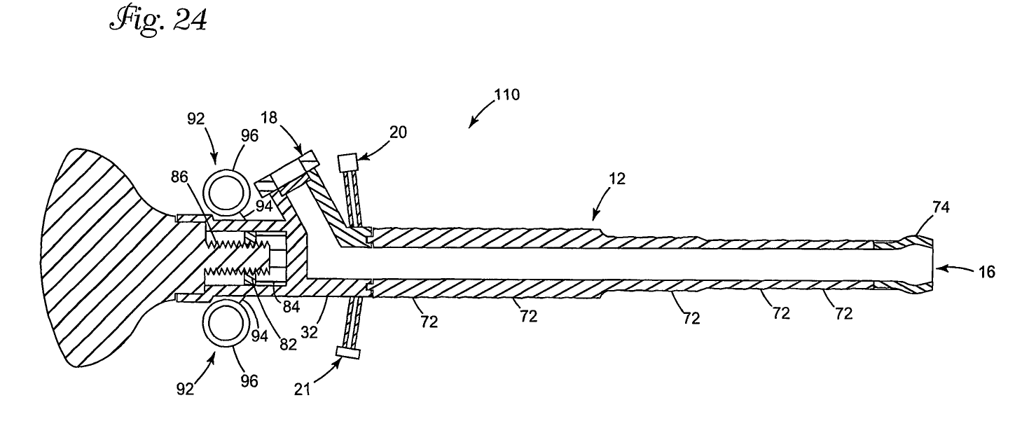

The stabilizer introducer 110 may further be equipped with any of a variety of additional control features. For example, with reference to FIGS. 23 and 24, the stabilizer introducer 110 may be equipped with one or more mechanisms 92 for selectively controlling the orientation of the distal member 74. More specifically, each mechanism 92 (if more than one is provided) includes, by way of example only, a ring-type handle member 96 coupled to a wire or cable 94 which, in turn, is coupled to a portion of the distal member 74. The cable 94s are preferably disposed within a lumen (not shown) formed through the housing 32 and the elongated body 12. With the proximal end of each cable 94 rigidly coupled to the handle member 96 and each of the distal ends rigidly coupled to the distal member 74, the surgeon may selectively alter the orientation of the distal opening 16 by manipulating (retracting or advancing) the handle member 96. In the embodiment shown, this may be easily accomplished by having the surgeon use his or her forefinger and middle finger in the respective ring-type handle members 96 and selectively pulling on the handles 96 to properly orient the distal opening 16 over the surgical site. Alternatively, there may be a single cable 94 and connection to the distal member 74, instead of the two cables 94 and connections as in the embodiment shown in FIGS. 23 and 24.

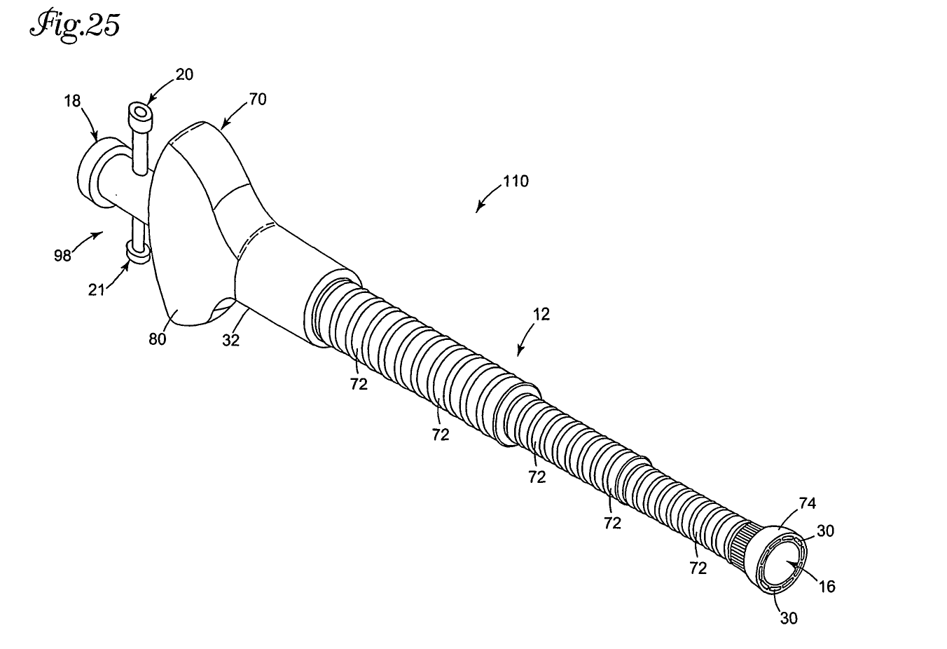

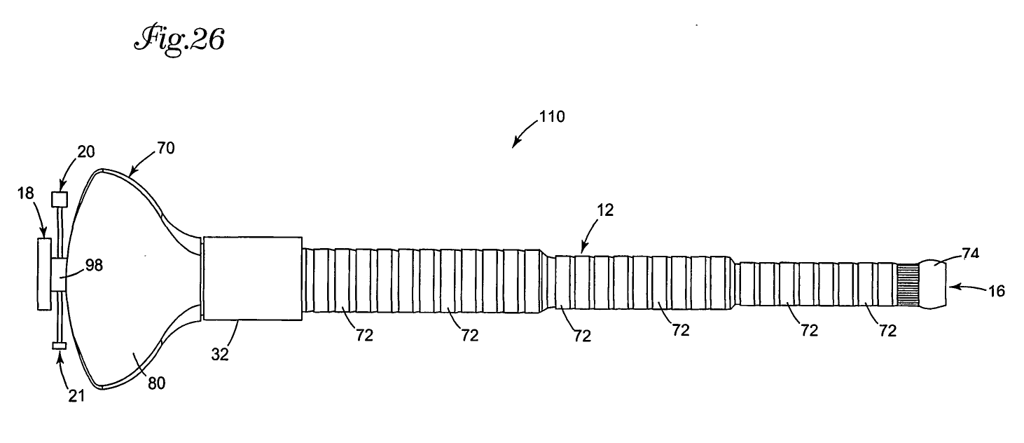

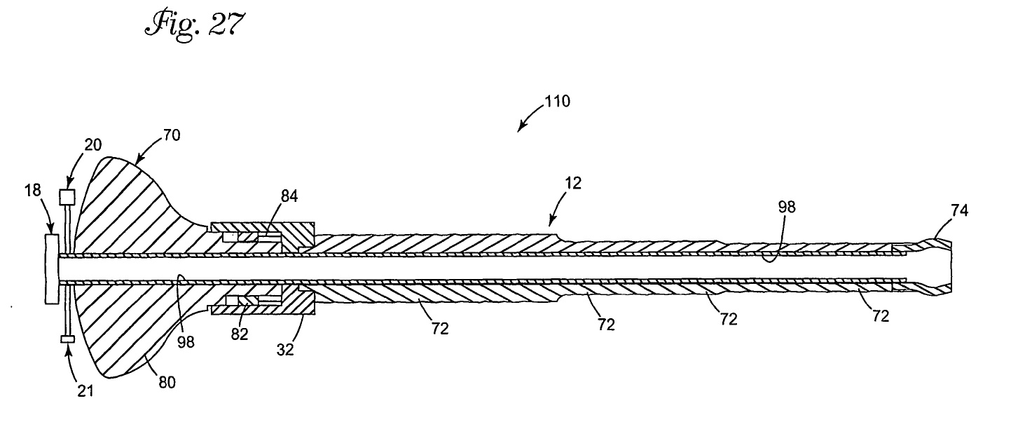

The stabilizer introducer 110 may also be constructed such that the stabilizing and introducer features are generally separable. For example, with reference to FIGS. 25-27, the stabilizer introducer 110 may include a separate introducer conduit 98 dimensioned to extend, in use, through the interior of the elongated body 12. The introducer conduit 98 is equipped with a hemostasis valve assembly 18 and a suction port assembly 20 at its proximal end and a distal member 74 at its distal end. A plurality of lumens (not shown) are preferably formed within the wall of the introducer conduit 98 for establishing fluid communication between the suction port assembly 20 and the additional suction ports 30 located on the contacting surface of the distal member 74. In this fashion, a suction source (not shown) coupled to the suction port 20 may be used to create a suction force at the ports 30 and thereby assist in maintaining the distal member 74 in position over the surgical site. The introducer conduit 98 may be constructed from any number of different biocompatible materials. However, in a preferred embodiment, the introducer conduit 98 will be constructed from a medical grade silicone-based plastic.

The introducer conduit 98 may be constructed such that the assemblies at the proximal end (hemostasis valve assembly 18 and suction port assembly 20) may be selectively removed from and coupled to the introducer conduit 98. In so doing, the introducer conduit 98 may be removed from the segments 72 forming the elongated body 12 following use and discarded. The stabilizer portion (including control mechanism 70 and elongated body 12) may be re-sterilized and equipped with a new introducer conduit 98. This advantageously minimizes cost, in that the more expensive stabilizer portion can be reused. The configuration of the introducer conduit 98 is also advantageous in that it allows the surgeon to introduce instruments and devices into the central lumen 22 in a generally straight fashion, rather than having to negotiate the angled hemostasis valve assembly 18 found in the embodiments shown in FIGS. 1-24. This may advantageously ease the task of controlling the instruments and devices when conducting beating heart bypass surgery according to the present invention.

Many other alterations or modifications may be made by those of ordinary skill in the art without departing from the spirit and scope of the invention. For example, although described above mainly in terms of “beating heart” surgery, it will be readily appreciated that the devices and methods of the present invention may be readily employed for “stopped heart” cardiac procedures, as well as alternative purposes such as cerebral surgery or any application whereby an occlusion must be bypassed.

The illustrated embodiments have been shown only for purposes of clarity and examples should not be taken as limiting the invention as defined by the following claims, which includes all equivalents, whether now or later devised.

1. A method of bypassing a region of restricted blood flow within a blood vessel, comprising the steps of:
providing a generally tubular structure having a first lumen and an open distal end;
positioning said generally tubular structure such that said open distal end is disposed over a region of restricted blood flow within a blood vessel;
filling the first lumen with a fluid;
creating an incision through the wall of said blood vessel over at least part of the region of restricted blood flow; and
deploying a bypass conduit such that the proximal and distal ends of the bypass conduit extend upstream and downstream of the region of restricted blood flow.

2. The method of claim 1 and further, wherein said generally tubular structure includes at least one suction lumen in communication with a vacuum source and at least one suction opening disposed proximate to the distal opening of the first lumen.

3. The method of claim 2 and further, wherein said generally tubular structure includes a shaping structure capable of maintaining the tubular structure in a predetermined shape during use.

4. The method of claim 3 and further, wherein said shaping structure comprises at least one shaping wire disposed within the wall of the tubular structure.

5. The method of claim 3 and further, wherein said shaping structure comprises a segmented stabilizer capable of being selectively shaped while in a first mode and then fixed in a rigid manner during a second mode.

6. The method of claim 5 and further, wherein said at least one suction lumen extends through the side walls of said segmented stabilizer, and said first lumen extends through a main lumen of said segmented stabilizer.

7. The method of claim 5 and further, wherein said generally tubular structure comprises a flexible conduit that, in use, extends through a main lumen of said segmented stabilizer and, after use, may be removed from the main lumen of the stabilizer.

8. The method of claim 5 and further, wherein at least one of the segmented stabilizer and generally tubular structure is equipped with at least one supplemental stabilizing member.

9. The method of claim 8 and further, wherein said at least supplemental stabilizing member has a contact surface that is generally co-planar with said distal opening of said first lumen.

10. The method of claim 9 and further, wherein said contact surface of said supplemental stabilizing member includes at least one suction opening in communication with a vacuum source.

11. The method of claim 1 and further, wherein said distal opening of said first lumen is one of circular and oval in shape.

12. The method of claim 7 and further, wherein said segmented stabilizer includes a control assembly for selectively going between said first mode and said second mode, said control assembly having a main lumen through which said flexible conduit extends in use.

13. The method of claim 12 and further, wherein said control assembly includes a twistable handle member, a slide member threadably coupled to the twistable handle member, and at least one wire member extending between the slide member and at least one segment forming part of the segmented stabilizer.

14. The method of claim 1 and further, wherein the generally tubular structure may be one of straight and curved in order to better position the open distal end of said first lumen over the region of restricted blood flow within said blood vessel.

15. The method of claim 5 and further, wherein at least one of said segmented stabilizer and said generally tubular structure includes a steering mechanism for selectively controlling the orientation of the open distal end of the first lumen.

16. The method of claim 15 and further, wherein said steering mechanism includes at least one control wire disposed within at least one of the side walls of the generally tubular structure and the segmented stabilizer, said at least one control wire having a handle member for manually controlling the orientation of the open distal end of the first lumen.

17. The method of claim 15 and further, wherein the steering mechanism comprises a robotically controlled arrangement capable of selectively controlling the orientation of the open distal end of the first lumen.

18. The method of claim 1 and further, wherein step (c) is accomplished from the exterior of the blood vessel by introducing a cutting element through the first lumen of the generally tubular structure.

19. The method of claim 18 and further, wherein a protective structural member is positioned within said region of restricted blood flow prior to said cutting step to ensure that only one vessel wall is cut.

20. The method of claim 19 and further, wherein said protective structural member is a catheter passed intravascularly such that at least a portion is disposed through said region of restricted blood flow.

21. The method of claim 1 and further, wherein step (c) is accomplished from the interior of the blood vessel by introducing a cutting element intravascularly into the region of restricted blood flow.

22. The method of claim 21 and further, wherein said cutting element is part of an intravascular catheter, said catheter being controllable to selectively advance the cutting element through the vessel wall once positioned within the region of restricted blood flow.

23. The method of claim 21 and further, wherein said intravascular catheter includes a debris catching element capable of being deployed downstream of said region of restricted blood flow to catch any debris that may be dislodged during said cutting step.

24. A surgical tool for introducing surgical instruments to a surgical site, the tool comprising:
an elongated body having a distal end to be positioned at the surgical site;
a first lumen longitudinally extending through the elongated body, the lumen having an opening at a distal end for access to the surgical site and having a hemostasis port at a proximal end for inserting surgical instruments partially into the first lumen and providing a seal around the partially inserted surgical instruments;
sealing mechanism associated with the elongated body to seal the distal end of the elongated body to the surgical site so that the distal opening of the first lumen is positioned and sealed over the surgical site, and wherein the lumen forms a sealed chamber for fluid.

25. The surgical tool of claim 24 and further, wherein said sealing mechanism includes at least one suction lumen in communication with a vacuum source and at least one suction opening disposed proximate to the distal opening of the first lumen.

26. The surgical tool of claim 25 and further, wherein said elongated body includes a shaping structure capable of maintaining the elongated body in a predetermined shape during use.

27. The surgical tool of claim 26 and further, wherein said shaping structure comprises at least one shaping wire disposed within the wall of the elongated body.

28. The surgical tool of claim 26 and further, wherein said shaping structure comprises a segmented stabilizer capable of being selectively shaped while in a first mode and then fixed in a rigid manner during a second mode.

29. The surgical tool of claim 28 and further, wherein said at least one suction lumen extends through the side walls of said segmented stabilizer, and said first lumen extends through a main lumen of said segmented stabilizer.

30. The surgical tool of claim 28 and further, wherein said elongated body comprises a flexible conduit that, in use, extends through a main lumen of said segmented stabilizer and, after use, may be removed from the main lumen of the stabilizer.

31. The surgical tool of claim 28 and further, wherein at least one of the segmented stabilizer and elongated body is equipped with at least one supplemental stabilizing member.

32. The surgical tool of claim 31 and further, wherein said at least supplemental stabilizing member has a contact surface that is generally co-planar with said distal opening of said first lumen.

33. The surgical tool of claim 32 and further, wherein said contact surface of said supplemental stabilizing member includes at least one suction opening in communication with a vacuum source.

34. The surgical tool of claim 24 and further, wherein said distal opening of said first lumen is one of circular and oval in shape.

35. The surgical tool of claim 30 and further, wherein said segmented stabilizer includes a control assembly for selectively going between said first mode and said second mode, said control assembly having a main lumen through which said flexible conduit extends in use.

36. The surgical tool of claim 35 and further, wherein said control assembly includes a twistable handle member, a slide member threadably coupled to the twistable handle member, and at least one wire member extending between the slide member and at least one segment forming part of the segmented stabilizer.

37. The surgical tool of claim 24 and further, wherein the elongated body may be one of straight and curved in order to better position the open distal end of said first lumen over the region of restricted blood flow within said blood vessel.

38. The surgical tool of claim 28 and further, wherein at least one of said segmented stabilizer and said elongated body includes a steering mechanism for selectively controlling the orientation of the open distal end of the first lumen.

39. The surgical tool of claim 38 and further, wherein said steering mechanism includes at least one control wire disposed within at least one of the side walls of the elongated body and the segmented stabilizer, said at least one control wire having a handle member for manually controlling the orientation of the open distal end of the first lumen.

40. The surgical tool of claim 38 and further, wherein the steering mechanism comprises a robotically controlled arrangement capable of selectively controlling the orientation of the open distal end of the first lumen.
